(19) United States
(12) Patent Application Publication
(10) Pub. No.: US 2024/0065944 A1
(43) Pub. Date: Feb. 29, 2024

(54) Biocompatible Enamel-Dentin Single-Component Adhesives

(71) Applicant: Ivoclar Vivadent AG, Schaan (LI)

(72) Inventors: Thomas Köhler, Reichenau (DE); Michael Barbisch, Klaus (AT); Thorsten Bock, Feldkirch (AT); Andreas Hartwig, Ritterhude (DE); Klaus Rischka, Tostedt (DE)

(21) Appl. No.: 18/457,018

(22) Filed: Aug. 28, 2023

(30) Foreign Application Priority Data
Aug. 31, 2022 (EP) 22193140.5

(51) Int. Cl. A61K 6/30; A61C 5/30; A61C 13/00; A61K 6/887; C08F 220/18

(52) U.S. Cl. CPC A61K 6/30; A61C 5/30; A61C 13/0024; A61K 6/887; C08F 220/1804

(57) ABSTRACT

A dental material having at least one copolymer obtained by (I) copolymerizing one or more monomers M_{G} having a polymerizable (meth)acrylate group, one or more OH group-containing (meth)acrylates M_{F}, one or more strongly acidic adhesive monomers M_{H} and optionally of one or more polymerizable carboxylic acid monomers M_{CS}, (II) reacting the resulting copolymer A₀ in a polymer-analogous reaction with a functionalized, radically polymerizable monomer M_{P}, which reacts with the OH groups of the monomer M_{F} in the polymer chain of A₀ to form a copolymer A_{P} having pendant polymerizable groups, and (III) neutralizing the copolymer Aₚ with a base. The material is particularly suitable as a dental adhesive.

### CROSS-REFERENCE TO RELATED APPLICATIONS

This application claims priority to European Patent Application No. 22193140.5 filed on Aug. 31, 2022, the disclosure of which is incorporated herein by reference in its entirety.

### TECHNICAL FIELD

The present invention relates to dental materials based on copolymers with adhesive properties, which are particularly suitable as adhesives for direct or indirect dental restorations.

### BACKGROUND

In dentistry, enamel-dentin adhesives are mainly used to create a strong and durable bond between direct or indirect restorative materials and the tooth structure (enamel and dentin). They usually contain a mixture of different radically polymerizable methacrylate monomers, polymers, an initiator system, solvents, such as aqueous ethanol or acetone, and other additives, such as stabilizers and rheology additives. The monomer mixture in turn usually contains a strongly acidic adhesive monomer, for example a methacryl group-containing dihydrogen phosphate with self-etching properties, which ensures strong bonding to the tooth structure, dimethacrylates, such as BisGMA and triethylene glycol dimethacrylate (TEGDMA), which promote rapid formation of a stable adhesive layer, and hydrophilic monomers, such as 2-hydroxyethyl methacrylate (HEMA), to promote penetration ability. Polymers, such as. methacrylated polyacrylic acid or itacon acid copolymers, are said to improve film formation of the adhesives and reduce technique sensitivity. Mixtures of camphorquinone (CQ) with a tertiary amine, such as 4-dimethylaminobenzoic acid ethyl ester (EMBO), are mainly used as polymerization initiators for visible light curing, which are combined with a redox initiator system in dual-cure systems.

U.S. Pat. No. 5,700,875, which is hereby incorporated by reference, discloses dental adhesives containing 60 to 98 wt % of a polymerizable monomer mixture, 2 to 40 wt % of polymer and 0.01 to 35 wt % of a polymerization initiator. The monomer mixture contains 2 to 25 wt % of acidic monomers with sulfonic acid, carboxylic acid, carboxylic anhydride or phosphoric acid groups. The polymer is a copolymer based on alkyl(meth)acrylates, styrene monomers, hydroxyalkyl (meth)acrylates, butadiene, polyalkyl methacrylates and/or polyvinyl acetate and is dissolved or dispersed in the monomer mixture. The adhesives are said to be easy to handle and have excellent adhesion.

R. R. Morães, J. W. Garcia, N. D. Wilson, S. H. Lewis, M. D. Barros, B. Yang, C. S. Pfeifer, J. W. Stansbury, J. Dent. Res. 91 (2012):179-184, investigate the effect of adding nanogel particles to dental adhesives. For this purpose, methacrylate functionalized nanogels of isobornyl methacrylate and UDMA or ethoxylated BisGMA with a particle size of 10 to 100 nm were dispersed in a BisGMA-HEMA-ethanol mixture at concentrations of 17 wt % and 23 wt %, respectively, and the mechanical properties and dentin adhesion were subsequently determined. Improved mechanical properties and dentin adhesion values were measured.

US 2013/0224692 A1, which is hereby incorporated by reference, discloses dental adhesives containing up to 15% by weight of a light-curing ionomer, radiopaque metal oxide nanoparticles, a phosphorus-containing acidic monomer, other monomers, water, a polar solvent and initiator. Ionomers are defined as polymers with ionic and radically polymerizable groups, such as polyalkenecarboxylic acids obtained by homo- or copolymerization of unsaturated mono-, di- or tricarboxylic acids and modified with monomers containing isocyanate groups.

EP 3 120 827 A1 and corresponding U.S. Pat. No. 9,833,389 B2, which is hereby incorporated by reference, disclose radically polymerizable dental materials suitable for use as dental adhesives, coating materials, filling composites and cements. The materials contain at least one radically polymerizable (meth)acrylate copolymer with strongly acidic phosphonic acid groups and a number-average molecular weight of 1,000 to 200,000 g/mol, radically polymerizable monomers with and without an acid group, and an initiator for the radical polymerization. The combination of acidic polymers and acidic monomers optimizes adhesion to enamel and dentin.

A disadvantage of known dental adhesives is that they usually have a more or less large residual monomer content after curing. Although the monomers used are not systemically toxic, they can nevertheless exhibit undesirable effects and lead to contact allergic reactions.

To improve biocompatibility, various concepts are proposed in the state of the art, especially bioadhesives inspired by shell adhesives and so-called ormocer-containing adhesives. Ormocers (organically modified ceramics) are cocondensates (heteropolysiloxanes) of di- or trialkoxysilanes containing organic groups, and possibly Ti or Zr alkoxides.

DE 196 43 007 A1 discloses adhesion promoters and adhesives based on reaction products of peptides or proteins from the byssus filaments of the blue mussel with radically polymerizable monomers, which are said to be suitable for dental purposes.

WO 2006/045034 A1 and corresponding US 2006084717 A1, which is hereby incorporated by reference, concern dental primers containing 0.1 to 50% by weight of DOPA (3,4-dihydroxyphenyl-L-alanine) in combination with dilute mineral acid. The DOPA is intended to allow simultaneous priming and etching of the tooth prior to application of a restorative material.

US 2011/0288252 A1, which is hereby incorporated by reference, discloses heteropolysiloxanes (ormocers), based on silanes containing dialkoxy and dihydroxyphenyl groups, modified with DOPA groups, for the preparation of adhesives. The heteropolysiloxanes can be cured by hydrolytic condensation with water or by radical polymerization of the polymerizable groups that may be present.

The disadvantage of such bioadhesives is their relatively low adhesive effect and the long period of time, often hours of days, required to achieve the full adhesive effect. They have therefore not found practical application as enamel-dentin adhesives.

Biocompatibility generally refers to the degree of tissue compatibility of a material used in the body or coming into contact with its surface. Biocompatible materials should behave neutrally towards the body and not lead to any negative effects. In particular, they must not have any toxicological, allergic, mutagenic, teratogenic or carcinogenic effects. Dental materials must not irritate oral and other body tissues.

DE 10 2018 204 655 A1 and corresponding US 2021161770 A1, which is hereby incorporated by reference, disclose glass ionomer cements containing an acid-reactive powder, a multi-proton acid, water and dispersed polymer particles. Preferred polymer particles are polyurethane particles. Preferably, these have an average particle size of less than 1 μm. Preferably, the particles are prepared as a primary or secondary dispersion. The polymer particles should be toxicologically safe and improve the cracking and fracture toughness of the cements. The materials preferably do not contain polymerizable compounds, such as (meth)acrylates.

By a primary dispersion, a skilled person understands a polymer dispersion that is obtained by first emulsifying monomers in water and then polymerizing them in the dispersed phase. Emulsifiers or protective colloids are required for the production of primary dispersions. To prepare a secondary dispersion, a polymer is prepared by solution polymerization in a volatile organic solvent. The polymer solution obtained is then dispersed in an aqueous phase and then the solvent is removed. Acidic or basic groups of the polymer are neutralized before dispersion. The salt groups formed during neutralization stabilize the polymer particles in the dispersion. Depending on the charge of the polymer particles, a distinction is made between anionic and cationic stabilized secondary dispersions. For the production of anionic secondary dispersions, for example, copolymers with acidic groups, e.g. carboxyl groups, are used, which are neutralized by the addition of bases. In this case, the stabilization of the secondary polymer dispersion is effected by carboxylate groups on the surface of the polymer particles. If copolymers with basic groups, such as tertiary amino groups, are used, cationic secondary dispersions are obtained, the neutralization being effected here by the addition of an acid.

Secondary dispersions are characterized by the fact that they require neither emulsifiers nor protective colloids. Emulsifiers increase the water vapor permeability and water sorption of polymer coatings. Y. Liu, W.-J. Soer, J. Scheerder, G. Satgurunathan, J.-L. Keddie, ACS Applied Materials & Interfaces 7 (2015) 12147-12157, have shown that emulsifier-free secondary dispersions of 2-ethylhexyl acrylate, acrylic acid, and n-butyl methacrylate exhibit lower water sorption and a lower water diffusion coefficient and thus better barrier properties than analogous coatings with a comparable emulsion polymer.

EP 0 305 850 A2 corresponding U.S. Pat. No. 5,041,495 A, which is hereby incorporated by reference, disclose aqueous secondary dispersions based on copolymers containing phosphate groups, which are obtained by polymerizing a mixture of 25 to 98% by weight of (meth)acrylic acid esters, 1 to 10% by weight of (meth)acrylic acid, 1 to 8% by weight of a phosphoric acid ester phosphate with a radically polymerizable group and optionally further olefinically unsaturated compounds. The dispersions are said to be suitable for the production of coatings filled with active pigments for corrosion protection.

EP 0 841 352 A1 relates to a process for the preparation of poly(meth)acrylate secondary dispersions which are said to have high storage stability. The dispersions are obtained by copolymerization of monoethylenically unsaturated monomers, such as acrylic or methacrylic acid alkyl esters, ethylenically unsaturated monomers bearing alcoholic hydroxyl groups, carboxyl group-containing unsaturated monomers, such as acrylic or methacrylic acid, and optionally a regulator in an organic solvent. After completion of the copolymerization, the amount of water-soluble residual monomers in the polymerization mixture is said to be less than 0.1 wt %. The dispersions are said be suitable as binders in water-dilutable coating compositions, for example for primers, top coats or clear varnishes for wood, metal or plastic coatings.

EP 1 270 619 A2 and corresponding U.S. Pat. No. 6,962,953 B2, which is hereby incorporated by reference, disclose aqueous vinyl polymer secondary dispersions containing at least one amphiphilic polymer composed of a hydrophobic segment and a hydrophilic segment. The amphiphilic polymer is intended to impart sufficient thermostability to the dispersions. The dispersions are prepared by polymerizing a mixture of ethylenically unsaturated monomers in the presence of an initiator in a water-solvent mixture with the addition of at least one amphiphilic polymer. The monomer mixture contains monoethylenically unsaturated monomers, such as acrylic or methacrylic acid alkyl esters, ethylenically unsaturated monomers bearing alcoholic hydroxyl groups, and carboxyl group-containing unsaturated monomers, such as acrylic or methacrylic acid. It may also contain monomers with sulfonic acid groups. The dispersions are said to be suitable as binders or binder components in water-dilutable coating compositions, for example for primers, fillers, top coats, clear varnishes, high gloss paints and one-coat paints, e.g. for industrial coatings, automotive OEM and refinish coatings.

B. Schlarb, M. G. Rau, S. Haremza, Prog. Org. Coat. 26 (1995) 207-215, have shown that by mixing two copolymers with different salt group contents, secondary dispersions can be prepared that allow the production of hydrophobic coatings. They combined a hydrophilic copolymer with salt groups with a hydrophobic copolymer without salt groups. The hydrophilic copolymer consists of 10 wt % acrylic acid, 53 wt % butyl acrylate and 37 wt % styrene, while the hydrophobic copolymer consists of 49 wt % n-butyl acrylate, 37 wt % styrene and 16 wt % methyl methacrylate. The coatings are said to be characterized by low water absorption and to be particularly suitable for the production of anti-corrosion paints and paints for road markings.

In a later publication, B. Schlarb, S. Haremza, W. Heckmann, B. Morrison, R. Müller-Mall, M. G. Rau MG, Prog. Org. Coat. 29 (1996) 201-208, the authors have shown that by varying the proportions and composition of the copolymers, polymer latex particles with core-shell structure can be prepared.

Ö. Kutlug, S. Reck, A. Hartwig, Int. J. Adhes. Adhes., 91 (2019) 36-42, describe crosslinkable pressure sensitive adhesives based on a secondary dispersion with two different copolymers. The dispersion contains a carboxy copolymer of 10 wt % acrylic acid and 90 wt % 2-ethylhexyl acrylate and an epoxy copolymer of 15 wt % glycidyl methacrylate, 20 wt % hydroxyethyl methacrylate and 65 wt % 2-ethylhexyl methacrylate. The adhesives are said to show improved crosslinking and, as a result, better shear strength.

### SUMMARY

It is an object of the present invention to provide biocompatible dental materials, which are particularly suitable as adhesives for dental applications, in particular as adhesives for cementing direct and indirect dental restorations to the tooth structure, i.e., to enamel and dentin. The dental materials should ensure good adhesion to the tooth structure, particularly under oral conditions.

### DETAILED DESCRIPTION

According to the invention, this object is achieved by dental materials containing at least one copolymer A_{P} obtainable by
(I) copolymerization of
   (a) one or more monomers M_{G} which comprise one (1) radically polymerizable (meth)acrylate group,
   (b) one or more OH group-containing (meth)acrylates M_{F},
   (c) one or more strongly acidic adhesive monomers M_{H} and
   (d) optionally one or more polymerizable carboxylic acid monomers M_{CS}, which comprise one (1) radically polymerizable group,
(II) reacting the copolymer A₀ obtained in step (I) in a polymer-analogous reaction with at least one functionalized, radically polymerizable monomer M_{P}, which reacts with the OH groups of the monomer M_{F} in the polymer chain of A₀ to form a copolymer A_{P} carrying pendant polymerizable groups, and
(III) neutralizing the copolymer Aₚ with a base.

In all cases, only monofunctional monomers are used to prepare the copolymer A₀, i.e., monomers with one radically polymerizable group. Polyfunctional monomers, i.e., monomers with two or more radically polymerizable groups, would cause crosslinking during polymerization and thus the formation of insoluble copolymers.

The copolymer A₀ preferably has the following composition:
(a) 20 to 80% by weight, more preferably 40 to 70% by weight, of monomer M_{G},
(b) 1 to 70% by weight, more preferably 5 to 40% by weight, of monomer M_{F},
(c) 1 to 40% by weight, more preferably 5 to 30% by weight, of monomer M_{H} and
(d) 0 to 30% by weight, more preferably 5 to 20% by weight, of monomer M_{CS},
   all percentages being based on the mass of the copolymer A₀. Copolymers in which the sum of (a), (b), (c) and (d) is 100% are particularly preferred.

The dental materials according to the invention are particularly suitable as dental restorative materials, especially as dental adhesives and more particularly as enamel-dentin adhesives, and are therefore also referred to as adhesives in the following for the sake of simplicity.

According to a preferred embodiment of the invention, copolymer Aₚ is prepared in the form of a secondary dispersion. For this, the copolymerization of the monomers M_{G}, M_{F}, M_{H} and optionally M_{CS} to copolymer A₀ according to step (I) is carried out in an organic solvent, after reaction of copolymer A₀ with monomer Mₚ (step (II)) and subsequent neutralization (step (III))
(IV) the copolymer solution obtained in step (III) is optionally mixed with water and stirred, and
(V) then the organic solvent is optionally distilled off.

The addition of water in step (IV) can be omitted if an aqueous solution of the base containing an amount of water sufficient to disperse the polymer solution is used for neutralization. The stirring in step (IV) is carried out so that the copolymer solution from step (III) is dispersed in the aqueous phase.

Preferably, (meth)acrylates are used as monomers M_{G}, which on homopolymeri-zation yield polymers with a glass transition temperature T_{G} of below 40° C. Methods commonly used in polymer analysis to determine the glass transition temperature T_{G} include differential scanning calorimetry (DSC) and dynamic mechanical analysis (DMA). Preferably, the glass transition temperature T_{G} is determined by DMA according to ISO-6721-11 from 2019.

Particularly preferred monomers M_{G} are alkyl and cycloalkyl (meth)acrylates, especially linear or branched C₁-C₂₅-alkyl (meth)acrylates and C₄-C₁₄-cycloalkyl (meth)acrylates, more preferably linear C₂-C₁₆-alkyl and C₆-C₁₀-cycloalkyl methacrylates and even more preferably linear C₂-C₁₀-alkyl methacrylates, in particular n-propyl methacrylate (T_{G}=35° C.), neopentyl methacrylate, n-butyl methacrylate (T_{G}=20° C.), phenylethyl methacrylate (T_{G}=26° C.), n-pentyl methacrylate, n-hexyl methacrylate (T_{G}=−5° C.), cyclohexyl methacrylate, 2-ethylhexyl methacrylate, n-octyl methacrylate (T_{G}=−20° C.), n-dodecyl methacrylate (T_{G}=−65° C.), tetradecyl methacrylate (T_{G}=−9° C.) and hexadecyl methacrylate (T_{G}=16° C.), and the corresponding acrylates and mixtures thereof, in all cases the methacrylates being preferred over the acrylates. Most preferred are n-butyl methacrylate, n-hexyl methacrylate, 2-ethylhexyl methacrylate and mixtures thereof.

Preferred OH group-containing (meth)acrylates M_{F} are monofunctional radically polymerizable monomers, particularly preferred are monofunctional monomers with one (1) OH group. More preferred are 2-hydroxyethyl (meth)acrylate, 2-hydroxypropyl (meth)acrylate, 3-hydroxypropyl (meth)acrylate, 4-hydroxybutyl (meth)acrylate and mixtures thereof. Even more preferred are 2- and 3-hydroxypropyl methacrylate (HPMA), especially 2-hydroxyethyl methacrylate (HEMA) and mixtures thereof.

The hydroxyl group of the monomers M_{F} permits polymer-analogous functionalization of the copolymer A₀ with polymerizable monomers M_{P}, which carry one (1) functional group capable of reacting with the hydroxyl group to form a chemical bond, and at least one radically polymerizable group. Preferred functional groups are isocyanate groups, preferred radically polymerizable groups are (meth)acrylate groups and in particular methacrylate groups.

Preferred monomers M_{P} are 2-isocyanatoethyl (meth)acrylate, the addition products of 1 mole of 2-hydroxyethyl (meth)acrylate or 2-hydroxypropyl (meth)acrylate each with 1 mole of a diisocyanate, preferably hexamethylene-1,6-diisocyanate (HMDI), 2,2,4-trimethyhexamethylene-1,6-diisocyanate (TMDI) or isophorone diisocyanate (IPDI), and mixtures thereof. A particularly preferred monomer M_{P} is 2-isocyanatoethyl methacrylate (IEMA).

The content of polymerizable groups in the copolymer A_{P} and thus the degree of crosslinking can be specifically adjusted by the content of the monomer building blocks M_{F} in the copolymer A₀ and the degree of reaction of the polymer-analogous reaction with the monomer(s) M_{P}. Preferably, the amount of monomer M_{P} is selected such that 10 to 100 mol %, preferably 30 to 100 mol % and more preferably 40 to 100 mol % of the OH groups of the monomer building blocks M_{F} are reacted with the monomer M_{P}.

Strongly acidic adhesive monomers M_{H} are radically polymerizable monomers which carry at least one strongly acidic group and one (1) radically polymerizable group. Preferred adhesive monomers M_{H} are monomers with a pKₐ value (at room temperature) of 0.5 to 4.0, preferably 1.0 to 3.5 and more preferably 1.5 to 2.5. Preferred strongly acidic groups are phosphoric acid and phosphonic acid groups, preferred radically polymerizable groups are (meth)acrylate groups and in particular methacrylate groups. Particularly preferred adhesive monomers M_{H} are radically polymerizable phosphoric acid esters, such as 2-(meth)acryloyloxypropyl dihydrogen phosphate, 2-(meth)acryloyloxyethyl dihydrogen phosphate, 4-(meth)acryloyloxybutyl dihydrogen phosphate, 6-(meth)acryloyloxyhexyl dihydrogen phosphate, 10-acryloyloxydecyl dihydrogen phosphate (ADP) and 10-methacryloyloxydecyl dihydrogen phosphate (MDP), and polymerizable phosphonic acids, such as vinyl phosphonic acid, 4-vinylphenyl phosphonic acid, 4-vinylbenzyl phosphonic acid, 2-(meth)acryloyloxyethyl phosphonic acid, 2-[4-(dihydroxyphosphoryl)-2-oxa-butyl]-acrylic acid ethyl ester (DHPBAE) and mixtures thereof. MDP, vinylphosphonic acid, DHPBAE and mixtures thereof are very particularly preferred.

Radically polymerizable carboxylic acid monomers M_{CS} are monomers containing at least one carboxylic acid group, preferably 1 to 2 carboxylic acid groups, and one (1) radically polymerizable group. Preferred radically polymerizable groups are vinyl, acrylic and vicinally or geminally substituted ethylene groups and in particular methacrylic groups. Ethylene groups may, for example, be vicinally or geminally substituted by —CH₃ and —COOH, —COOH and —CH₂ —COOH or two COOH groups. Preferred monomers M_{CS} are (meth)acrylic acid, itaconic acid, crotonic acid, maleic acid and fumaric acid and their half esters, 2-(hydroxymethyl)acrylic acid, 4-(meth)acryloyloxyethyltrimellitic acid, 10-(meth)acryloyloxydecylmalonic acid, 2-(meth)acryloyloxymethylsuccinic acid, 4-vinylbenzoic acid and mixtures thereof. Acrylic acid and especially methacrylic acid (MAA) are particularly preferred.

Preferred according to the invention are adhesives which, in addition to the copolymer A_{P}, contain at least one second copolymer B_{P} whose composition differs from that of the copolymer A_{P}, preferably a copolymer obtainable by
(I) copolymerization of
   (a) one or more monomers M_{G} with one (1) polymerizable (meth)acrylate group,
   (b) one or more OH group-containing (meth)acrylates M_{F}, and
   (c) optionally one or more polymerizable carboxylic acid monomers M_{CS},
(II) reacting the resulting copolymer B₀ in a polymer-analogous reaction with at least one functionalized, radically polymerizable monomer M_{P}, which reacts with the OH groups of the monomer M_{F} in the polymer chain of B₀ to form a copolymer B_{P} carrying pendant polymerizable groups, and
(III) neutralizing the copolymer Bₚ with a base.

The copolymer B₀ preferably has the following composition:
(a) 30 to 85% by weight, more preferably 40 to 80% by weight, of monomer M_{G},
(b) 5 to 55% by weight, more preferably 10 to 50% by weight, of monomer M_{F} and
(c) 1 to 40% by weight, more preferably 5 to 30% by weight, of monomer M_{CS},
   all percentages being based on the mass of the copolymer B₀. Copolymers in which the sum of (a), (b) and (c) is 100% are particularly preferred.

Copolymer Bₚ is preferably also prepared in the form of a secondary dispersion. The secondary dispersion of copolymer B₀ is prepared in the same way as copolymer A₀ by copolymerization of the monomers M_{G}, M_{F} and, if desired, M_{CS} in an organic solvent (step (I)). The resulting copolymer B₀ is then reacted with the monomer Mₚ in a polymer-analogous reaction to give the copolymer B_{P} (step (II)). The amount of monomers M_{P} is preferably selected such that 10 to 100 mol %, preferably 30 to 80 mol % and particularly preferably 40 to 60 mol % of the OH groups of the monomer building blocks M_{F} react with a monomer M_{P}. Subsequently, the copolymer B can be neutralized by adding a base (step (III)), water can optionally be added to the copolymer solution obtained (step (IV)), and then the organic solvent can optionally be distilled off (step (V)). The obtained secondary dispersion of copolymer B_{P} can then be mixed with a secondary dispersion of copolymer A_{P}. Secondary dispersions containing two or more different copolymers are referred to herein as secondary co-dispersions. Preferably, the monomers M_{G}, M_{F}, M_{P} and optionally M_{CS} described above are used to prepare copolymer B_{P}, and the same or different monomers as used to prepare A_{P} can be used to prepare B_{P}. The copolymer B_{P} does not contain any strongly acidic monomers M_{H}, or repeating units derived therefrom. In all cases, only monofunctional monomers, i.e. monomers with one radically polymerizable group are used to prepare the copolymer B_{P}.

According to a preferred embodiment of the invention, the secondary co-dispersions are prepared by preparing the solutions of the copolymers A_{P} and B_{P} separately and mixing them with each other before neutralization and only then neutralizing them by adding a base. Water is then optionally added to the neutralized copolymer dispersion, if necessary, and then the organic solvent is optionally distilled off.

Preferred bases for neutralizing the acidic monomer building blocks of the copolymers Aₚ, B_{P} or of the mixture of A_{P} and B_{P} are LiOH, KOH, NaOH, NH₄OH and organic amines, preferably ethylamine, n-butylamine, dibutylamine, trimethylamine, tributylamine (TBA), triethylamine (TEA), N-methylmorpholine, or dimethylcyclohexyl amine, amino acid derivatives, preferably lysine methyl ester dihydrochloride, arginine hydrochloride, or glycine ethyl ester, OH-functionalized amines, preferably methyldiethanolamine, diethylethanolamine, dimethylethanolamine, tris(hydroxy-methyl)aminomethane, ethanolamine, diethanolamine, triethanolamine (TEOA), butanolamine, dibutanolamine, 3-dimethylamino-2-propanol (DMAP) or 1,3-diamino-2-propanol (DAP), higher molecular weight ether group-containing amines and mixtures thereof. Preferred higher molecular weight ether group-containing amines are amino group-terminated oligomeric ethylene oxide or propylene oxide oligomers. Such polyetheramines are commercially available, for example, under the name JEFFAMINE®. The bases mentioned are preferably used in the form of aqueous solutions. Particularly preferred are aqueous solutions of DMAP, TBA, TEA, DAP and mixtures thereof.

The acidic groups of the copolymers A_{P} and B_{P} can be neutralized completely or preferably partially. Preferably, the base or base mixture is added in an amount of from 20 to 90 mol %, more preferably from 25 to 80 mol % and even more preferably from 30 to 75 mol %, based on the content of strongly acidic adhesive monomers M_{H}. When using a mixture of A_{P} and B_{P}, the neutralization is preferably carried out after mixing the copolymers.

The particle size of the copolymer particles in the secondary dispersion formed can be specifically adjusted by the type of bases used and the degree of neutralization. According to the invention, particles with an average particle size of 1 to 700 nm, in particular of 5 to 500 nm and very especially of 20 to 300 nm are preferred.

Unless otherwise stated, all particle sizes herein are volume averaged particle sizes (D50 values), i.e. 50% of the total volume of all particles is contained in particles having a diameter smaller than the stated value. The D10 value is accordingly the volumetric diameter at which 10% of the total filler volume is smaller than the specified value.

Particle size determination in the range from 0.1 μm to 1000 μm is preferably carried out by means of static light scattering (SLS), for example with an LA-960 static laser scattering particle size analyzer (Horiba, Japan) or with a Microtrac S100 particle size analyzer (Microtrac, USA). Here, a laser diode with a wavelength of 655 nm and an LED with a wavelength of 405 nm are used as light sources. The use of two light sources with different wavelengths enables the measurement of the entire particle size distribution of a sample in only one measurement run, whereby the measurement is carried out as a wet measurement. For this purpose, an aqueous dispersion of the filler is prepared and its scattered light is measured in a flow cell. The scattered light analysis for calculating particle size and particle size distribution is carried out according to the Mie theory according to DIN/ISO 13320. The measurement of the particle size in a range from 1 nm to 0.1 μm is preferably carried out by dynamic light scattering (DLS) of aqueous particle dispersions, preferably with a He—Ne laser with a wavelength of 633 nm, at a scattering angle of 90° and at 25° C., e.g. with a Malvern Zetasizer Nano ZS (Malvern Instruments, Malvern UK).

In the case of aggregated and agglomerated particles, the primary particle size can be determined from TEM images. Transmission electron microscopy (TEM) is preferably performed using a Philips CM30 TEM at an accelerating voltage of 300 kV. For sample preparation, drops of particle dispersion are applied to a 50 Å thick copper grid (mesh size 300 mesh) coated with carbon, followed by evaporation of the solvent. The particles are counted and the arithmetic mean is calculated.

The adhesives according to the invention preferably contain, in addition to the copolymer(s) A_{P} and optionally B_{P}, at least one dispersion medium, particularly preferably ethanol, isopropanol, tert-butyl alcohol, water or a mixture thereof, most preferably ethanol, water or a mixture thereof. Most preferred is water.

Furthermore, the adhesives may contain one or more initiators for the radical polymerization, preferably a photoinitiator, in particular camphorquinone in combination with 4-dimethylaminobenzoic acid ethyl ester. However, the polymerization of the adhesives can also take place, for example, together with the polymerization of a radically curable restorative material that is applied to the adhesive. It is assumed that in this case initiator components and/or radicals diffuse from the restorative material into the adhesive layer and induce curing of the adhesive. The addition of an initiator is therefore optional, and adhesives that do not contain an initiator for radical polymerization are particularly preferred.

In addition, the adhesives may contain one or more fillers and/or one or more additives.

Preferably, the adhesives according to the invention have the following composition:
10 to 50% by weight, preferably 15 to 45% by weight and more preferably 20 to 35% by weight of at least one copolymer A_{P} and optionally at least one copolymer B_{P} and
50 to 90% by weight, preferably 55 to 85% by weight, and more preferably 65 to 80% by weight dispersion medium.

The adhesives preferably contain
10 to 70% by weight, preferably 15 to 60% by weight, and more preferably 20 to 55% by weight water.

The adhesives can also contain
0.001 to 10% by weight, preferably 0.01 to 8% by weight and more preferably 0.1 to 5% by weight of initiator for the free radical polymerization.

Further preferred are adhesives that contain
0 to 10% by weight, preferably 0 to 5% by weight and more preferably 0 to 3% by weight of filler and/or
0 to 5% by weight, preferably 0 to 3% by weight and more preferably 0 to 2% by weight of one or more additives.

All quantities given relate in each case to the total mass of the adhesive, the water content being included in the quantity of dispersion medium, if present. The adhesives according to the invention are preferably present in single-component form.

The amount of dispersion medium is measured so that the adhesives have the desired viscosity, i.e. are easy to apply and can be spread well on the substrate to be bonded, e.g. the tooth.

Preferred Initiators for the preparation of the adhesives are photoinitiators such as benzophenone, and benzoin and derivatives thereof, as well as α-diketones and derivatives thereof, such as 9,10-phenanthrenequinone, 1-phenylpropane-1,2-dione, diacetyl or 4,4′-dichlorobenzil. Preferred photoinitiators are camphorquinone and 2,2-dimethoxy-2-phenyl-acetophenone and especially α-diketones in combination with amines as reducing agents, such as 4-(dimethylamino)-benzoic acid ester, N,N-dimethylaminoethyl methacrylate, N,N-dimethyl-sym.-xylidine or triethanolamine. Also preferred are Norrish type I photoinitiators, in particular acyl of bisacylphosphine oxides, monoacyltrialkyl and diacyldialkylgermanium compounds, such as benzoyltri-methylgermanium, dibenzoyldiethylgermanium and bis-(4-methoxybenzoyl)diethyl-germanium. Further preferred are mixtures of different photoinitiators, such as dibenzoyldiethylgermanium in combination with camphorquinone and 4-dimethylaminobenzoic acid ethyl ester. Most preferred is camphorquinone in combination with 4-dimethylaminobenzoic acid ethyl ester.

Preferred fillers are organic or inorganic filler particles. Preferred inorganic particulate fillers are amorphous spherical materials based on oxides, such as SiO₂ or ZrO₂, or mixed oxides of SiO₂ and ZrO₂. Nanoparticulate fillers, such as pyrogenic silica or precipitated silica with an average primary particle size of 5 to 200 nm and preferably of 10 to 50 nm are particularly preferred. Fillers serve to improve the mechanical properties and/or to adjust the viscosity and are preferably added before the dispersion is formed. The adhesives may contain one or more fillers.

Other suitable additives include stabilizers, microbicidal agents and fluoride ion-releasing additives.

Surprisingly, it was found that the adhesives according to the invention exhibit high adhesion to the natural tooth structure and thus enable secure attachment of direct and indirect restorations to teeth. However, they are also suitable for bonding restorations together and for repairing dental restorations. The copolymers A_{P} and B_{P} carry radically polymerizable groups that enable crosslinking and curing of the adhesives and ensure good mechanical properties. In addition, these groups can react with radically polymerizable groups of, for example, a dental filling material and thus improve its adhesion.

It should be noted that the adhesives exhibit high and durable adhesion even under oral conditions. This is particularly surprising because both copolymer A_{P} and copolymer B_{P} are composed to a considerable extent of hydrophilic monomers, so that the adhesive layers formed from them should be hydrophilic and thus sensitive to moisture. The inventors have found that by selectively choosing the type and amount of monomers used to prepare the copolymers A_{P} and B_{P} and by selectively incorporating polymerizable side groups into the copolymer chains, it is possible to obtain adhesives that ensure stable adhesion even under moist conditions and are thus eminently suitable for dental applications and, in particular, for intraoral use.

A particular advantage of the adhesives according to the invention is that they contain no monomers and, in particular, no radically polymerizable monomers, which is advantageous from a toxicological point of view. The adhesives are therefore characterized by high biocompatibility. In addition, the adhesives preferably also do not contain emulsifiers and surfactants.

Biocompatibility was investigated by standardized test methods using aqueous extracts of the cured adhesives. The extracts showed no cytotoxicity and no genotoxicity to mouse fibroblasts.

A preferred process for preparing the copolymers of the invention is described below.

The copolymers A_{P} and B_{P} are synthesized by radical polymerization of the monomers M_{G}, M_{F}, and optionally M_{H} and M_{CS}, preferably by solution polymerization in a readily volatile, water-miscible organic solvent that dissolves poly(meth)acrylates well. Preferred solvents are acetone (boiling point: 56° C.), methyl ethyl ketone (80° C.), diethyl ketone (102° C.), methyl isobutyl ketone (116° C.), tetrahydrofuran (66° C.) and acetonitrile (82° C.) and mixtures thereof. Acetone and methyl ethyl ketone are particularly preferred. The monomers are preferably used in a total monomer concentration of 10 to 60% by weight, preferably 15 to 50% by weight and more preferably 20 to 40% by weight, based on the mass of the reaction mixture.

The polymerization is preferably carried out in the presence of an initiator for the radical polymerization. Preferred polymerization initiators for the preparation of the copolymers are thermal initiators such as azo compounds, e.g. 2,2′-azobis(isobutyronitrile) (AIBN), 2,2′-azobis(2-methylbutyronitrile), 4,4′-azobis(4-cyanovaleric acid), dimethyl-2,2′-azobisbutyrate, 1,1′-azobis(1-cyclohexanecarbonitrile) or 2,2′-azobis(2,4-dimethyl-4-methoxyvaleronitrile), peroxides, e.g. dilauryl peroxide, tert-butylperoxy-2-ethyl hexanoate, tert-butyl peroxybenzoate or di-tert-butyl peroxide, dicumyl peroxide, peroxidicarbonates, e.g. dicyclohexyl peroxidicarbonate or bis-(4-tert-butylcyclohexyl) peroxidicarbonate. Particularly preferred are AIBN, tert-butylperoxy-2-ethylhexanoate, tert-butyl peroxybenzoate or di-tert-butyl peroxide. In order to achieve complete monomer conversion, it is preferred to post-dose the initiator in the course of the polymerization. Optionally, post-polymerization can be carried out at elevated temperature. The total initiator concentration used is preferably in the range from 0.1 to 5.0% by weight, particularly preferably from 0.5 to 4.0% by weight and most preferably from 0.7 to 2.5% by weight, based on the mass of the reaction batch. Any residual monomers present are preferably removed by dialysis or reprecipitation of the copolymers.

The number-average molecular weight Mₙ of the polymers formed increases with increasing monomer concentration and decreasing initiator concentration. A chain regulator can be added to selectively adjust the molar mass, even at high monomer concentrations. Preferred chain regulators are thiols, e.g. mercaptoethanol, tert-dodecylmercaptan or laurylmercaptan, disulfides, such as diisopropyl xanthogen disulfide. Particularly preferred chain regulators are amino acid derivatives containing thiol groups such as N-acetyl-(L)-cysteine (AC), N-acetyl-L-cysteine methyl ester, N-acetyl-L-cysteine ethyl ester, most preferred is AC. Amino acid derivatives containing thiol groups yield polymers with particularly good biocompatibility. Chain regulators are preferably used in a concentration of 0.1 to 5.0% by weight, more preferably 0.5 to 4.0% by weight and even more preferably 0.7 to 3.0% by weight, based on the mass of the reaction mixture.

Initiators and chain regulators are incorporated into the polymer as end groups during polymerization. As is usual in polymer chemistry, these are not taken into account when specifying the polymer composition.

The number-average molar mass Mₙ of the copolymers A₀ and B₀ is preferably in a range from 5,000 to 60,000 g/mol, more preferably from 10,000 to 50,000 g/mol and most preferably from 15,000 to 40,000 g/mol, the number-average molar mass Mₙ being determined by gel permeation chromatography (GPC).

Gel permeation chromatography (GPC) is a relative method in which molecules are separated based on their size, or more precisely, their hydrodynamic volume. The absolute molar mass is determined by calibration with known standards. Preferably, narrowly distributed polystyrene standards are used as calibration standards. These are commercially available. Styrene-divinylbenzene columns are used as separation material and tetrahydrofuran (THF) as eluent. Styrene-divinylbenzene columns are suitable for organic soluble synthetic polymers. The measurement is carried out with diluted solutions (0.05-0.2% by weight) of the polymers to be investigated.

Alternatively, the number-average molar mass can be determined by the well-known methods of freezing point depression (cryoscopy), boiling point elevation (ebullioscopy) or from the depression of the vapor pressure (vapor pressure osmometry). These are absolute methods that do not require calibration standards. Concentration series of 4 to 6 dilute polymer solutions with concentrations of 0.005 to 0.10 mol/kg are examined and then the measured values are extrapolated to a concentration of 0 mol/kg.

Subsequently, the copolymers are reacted with the monomer M_{P} in a polymer-analogous reaction, preferably with 2-isocyanatoethyl methacrylate (IEMA). The reaction is preferably carried out in the copolymer solution from the first step. For this, the monomer Mₚ, e.g. IEMA, is dissolved in an organic solvent, preferably in acetone, methyl ethyl ketone, diethyl ketone, methyl isobutyl ketone, tetrahydrofuran or acetonitrile, particularly preferably in acetone, and is dropped to the polymer solution at a temperature of 20 to 60° C., preferably 40 to 55° C. To accelerate the reactions, catalysts such as tin (II) oxalate or octanoate, dibutyltin dilaurate, tri-o-tolyl bismuth, triphenyl bismuth, tris-(2-methylphenyl) bismuth, tris-(2-methoxyphenyl) bismuth or bismuth (III) neodecanoate can be added. Particularly preferred are bismuth catalysts, such as tri-o-tolyl bismuth or bismuth (III) neodecanoate, which are characterized by high biocompatibility. According to a particularly preferred embodiment, the reaction with the monomer M_{P} is carried out without a catalyst.

Subsequently, the acidic groups of copolymers Aₚ and B_{P} are neutralized by adding a base. For the preparation of secondary co-dispersions, the copolymers A_{P} and B_{P} are preferably mixed together before neutralization. One or more bases are added to the copolymer Aₚ or the mixture of A_{P} and B_{P}, dissolved in an organic solvent which is at least partially miscible with water and has a lower boiling temperature than water, or in an azeotrope whose boiling point is lower than that of water. Preferably, the base is added stepwise in the form of an aqueous solution.

If the amount of water in the base solution is not sufficient to disperse the copolymer solution, or if the base is not used in the form of an aqueous solution, water is added to the neutralized copolymer solution. The mixture is preferably stirred intensively and the organic solvent is then preferably at least partially evaporated. When the solvent evaporates, a viscous secondary dispersion or secondary co-dispersion is formed. The secondary dispersion or secondary co-dispersion obtained contains particles of the copolymer(s). The secondary dispersion or secondary co-dispersion can be used directly as an adhesive or to prepare the adhesive.

The advantageous properties of the dental materials according to the invention are largely due to the radically polymerizable copolymers A_{P} and optionally B_{P}. These are present in the form of particles which enable the preparation of stable dispersions. The copolymers carry acidic groups which, after neutralization, cause anionic stabilization of the dispersions. At the same time, the combination of strongly acidic monomers and carboxylic acid monomers results in high adhesion to dentin and enamel. After application of the adhesives to the tooth surface and subsequent drying, the copolymers form a well-adherent polymer film, which enables a strong bond with dental restorative materials such as restorative composites via the polymerizable groups of the monomer M_{P}. The copolymers A_{P} and, if present, B_{P} enable the preparation of one-component, monomer-free adhesives with high biocompatibility.

Copolymers suitable according to the invention can preferably be prepared in the manner described above. However, the copolymers A_{P} and B_{P} can also be synthesized by other known methods of radical polymerization, for example by substance polymerization, precipitation polymerization, suspension or emulsion polymerization and the living free-radical polymerization methods known in the prior art. The copolymers obtained in these processes can be dissolved in an organic solvent that is at least partially miscible with water and has a boiling temperature below that of water, and reacted with a polymerizable monomer M_{P} by polymer-analogous reaction. The polymer solution is then dispersed in water with vigorous stirring, the acid groups are neutralized wholly or partially with a base before, after or during dispersion in water, and finally the organic solvent is substantially evaporated. The copolymers can then be dispersed in a suitable dispersing agent.

The materials according to the invention are particularly suitable as dental materials for intraoral use by the dentist for the restoration of damaged teeth (therapeutic application), especially as dental adhesives and in particular as enamel-dentin adhesives.

The materials according to the invention can also be used as extraoral materials (non-therapeutic), for example as adhesives in the fabrication or repair of dental restorations. They are also suitable as adhesives for the fabrication and repair of inlays, onlays, crowns or bridges.

For application, the adhesives according to the invention are preferably applied to the prepared enamel and/or dentin surface with a brush and possibly rubbed onto it. Film formation can be accelerated by evaporation of the liquid, e.g. by blowing with air or by heating with an artificial heat source or radiation. In this process, an adherent polymer film is formed. The enamel and/or dentin surface can be preconditioned beforehand by usual measures, such as etching with 37% phosphoric acid and/or drying with an air stream. However, this is not necessary and excellent adhesion is achieved even without acid pretreatment. Subsequently, a light-curing restorative composite, for example, can be applied and cured by irradiation with light. If the adhesive contains a photoinitiator, the adhesive layer can be cured by irradiation before the restorative composite is applied.

With the adhesives according to the invention, shear bond strengths on dentin and enamel of 15 MPa or more can be achieved, which ensures a very good and durable bond between the restorative material and the tooth structure.

The invention is explained in more detail in the following examples. The examples demonstrate that the enamel-dentin single-component adhesives according to the invention are biocompatible and lead to good enamel-dentin bonding.

### EXAMPLES

### Example 1

Synthesis of Copolymer A_{P} by Controlled Free Radical Copolymerization in Solution and Functionalization with 2-Isocyanatoethyl Methacrylate (EMA).

### a) Copolymerization in Solution:

In a beaker, 55 g of n-butyl methacrylate (BuMA), 15 g of 2-hydroxyethyl methacrylate (HEMA), 20 g of MDP und 10 g of methacrylic acid (MAA) were dissolved in 100 g of acetone using an ultrasonic bath. In a second beaker, 1 g of AIBN and 1 g of N-acetylcysteine were dissolved in 50 g of acetone with stirring. The two solutions were combined and transferred to the large dropping funnel of an apparatus consisting of a 500 ml three-neck flask equipped with a reflux condenser, 2 dropping funnels (50 ml and 250 ml), nitrogen feed (on large dropping funnel), bubble counter (on reflux condenser) and a large magnetic stir bar. In the three-neck flask, 100 g of acetone was introduced. Then, about 25% by volume of the acrylate solution was let into the three-neck-flask. The entire apparatus was intensively purged with nitrogen for 15 min. After that, the nitrogen flow was controlled down so that one bubble per second was visible in the bubble counter. Under nitrogen flow, the solution was refluxed in the three-neck flask for 30 min (80° C. oil bath temperature), after which the solution remaining in the dropping funnel was added to the boiling polymerization solution within 90 min. After addition, 1 g AIBN dissolved in 50 g acetone was again added rapidly to the boiling solution via the small dropping funnel. After a total of 7 h polymerization time, the solution polymerization was terminated by switching off the apparatus and cooling. A clear, slightly viscous solution of copolymers A₀ was obtained.

b) Functionalization of A₀ with IEMA (=M_{P}) to Form the Copolymer A_{P}

The polymer solution of A₀ obtained above was transferred to a 1 l three-neck flask equipped with a 100 ml dropping funnel and a reflux condenser. A solution of 17.84 g IEMA dissolved in 50 g acetone was prepared and transferred to the dropping funnel. At a temperature of 50° C., the IEMA solution was added to the polymer solution within one hour. The solution was then heated to 50° C. for another 3 h. To complete the reaction, the obtained solution was then stirred for another 14 h at room temperature. The complete conversion of the isocyanate was monitored by FTIR spectroscopy (NCO band). The amount of IEMA used corresponded to a complete conversion of the HEMA building blocks in A₀. After the reaction was completed, the solution was very viscous, so the solution of the copolymer A_{P} was diluted with 100 g acetone.

### Example 2

Synthesis of Copolymer be by Controlled Free Radical Copolymerization in Solution and Functionalization with IEMA.

### a) Copolymerization in Solution:

In one beaker, 60 g BuMA, 30 g HEMA and 10 g MAA were dissolved in 100 g acetone. In a second beaker, 1 g AIBN and 1 g N-acetylcysteine were dissolved in 50 g acetone with stirring. The two solutions were combined and transferred to the large dropping funnel of an apparatus consisting of a 500 ml three-neck flask equipped with a reflux condenser, 2 dropping funnels (50 ml and 250 ml), nitrogen feed (on large dropping funnel), bubble counter (on reflux condenser) and a large magnetic stir bar. In the three-neck flask, 100 g of acetone was introduced. Then, about 25% by volume of the methacrylate solution was let into the three-neck flask. The entire apparatus was intensively purged with nitrogen for 15 min. After that, the nitrogen flow was controlled down so that one bubble per second was visible in the bubble counter. Under nitrogen flow, the solution was refluxed in the three-neck flask for 30 min (80° C. oil bath temperature), after which the solution remaining in the dropping funnel was added to the boiling polymerization solution within 90 min. After addition, 1 g AIBN dissolved in 50 g acetone was again added rapidly to the boiling solution via the small dropping funnel. After a total of 7 h polymerization time, the solution polymerisation was terminated by switching off the apparatus and cooling. A clear solution of copolymer B₀ was obtained.

b) Functionalization of B₀ with IEMA (=M_{P}) to Form the Copolymer B_{P}

The polymer solution of B₀ obtained above was transferred to a 1 l three-necked flask equipped with a 100 ml dropping funnel and a reflux condenser. A solution of 17.84 g IEMA dissolved in 50 g acetone was prepared and transferred to the dropping funnel. At a temperature of 50° C., the IEMA solution was added to the polymer solution within one hour. Subsequently, the solution was heated to 50° C. for another 18 h. The complete conversion of the isocyanate was monitored by FTIR spectroscopy (NCO band). The amount of IEMA used corresponded to a complete conversion of the HEMA building blocks in B₀. After completion of the reaction, a slightly yellowish, clear solution of the copolymers B_{P} was obtained.

### Example 3

Preparation of a Secondary Co-Dispersion Based on A_{P} and B_{P} (70% Neutralization)

About ⅛ of the solution of copolymer A_{P} obtained in Example 1 and about ⅛ of the solution of copolymer B_{P} obtained in Example 2 were weighed separately into a beaker each. The two solutions were mixed in a 250 ml round bottom flask with vigorous stirring. To the resulting turbid solution, 560 mg of dimethylamino-1-propanol (DMAP) dissolved in 12.5 g of demineralized water was added briskly in small portions using a glass pipette. The amount of DMAP corresponds to the amount required to neutralize 70% of the phosphoric acid groups in Copolymer A_{P}. After the addition was completed, a clear solution was present. The acetone was removed from the solution on a rotary evaporator at a pressure of about 250 mbar and a water bath temperature of about 40° C. After removal of the organic solvent, a secondary co-dispersion was obtained as a milky, viscous liquid.

### Example 4

### Preparation of an Adhesive Based on the Secondary Co-Dispersion from Example 3 and Measurement of Shear Bond Strength to Tooth Structure

To 6 g of the secondary co-dispersion obtained in Example 3, the photoinitiator components camphorquinone (CQ: 45 mg) and ethyl 4-(dimethylamino)benzoate (EMBO: 16 mg), both dissolved in a total of 4 g of ethanol, were added and the mixture was mixed by vigorous shaking to form a clear solution. The solid content of the mixture was 30% by weight. The mixture was used as an adhesive for shear adhesion measurements.

For shear bond strength measurements on enamel and dentin, according to ISO 29022: 2013 (Dentistry-Adhesion-Notched-edge shear bond strength test), bovine teeth were embedded in a silicone mold made of Dublisil 15 (addition-crosslinked vinyl polysiloxane, Dreve Company) with an embedding agent/casting resin (Visco-Voss GTS, Vosschemie Company, or Bosworth Fast Tray) so that the labial side of the bovine substrate was on the underside/bottom of the silicone mold and the bovine substrate was covered with 2 to 5 mm of embedding/casting resin. The teeth covered with resin were de-aerated together with the mold in a vacuum oven at approx. 400 mbar for approx. 30 s. Curing took place overnight at room temperature and ambient pressure. The next day, the test specimens were removed from the silicone mold. The tooth surfaces, which had been ground with abrasive paper (grit size 120 and 400), were rinsed with lukewarm water and pre-tempered to 37° C. The dentin or enamel surfaces were blotted dry (Kimtech Wipes). All dentin and enamel shear bond strength values were determined without prior etching of the teeth with H₃PO₄ in the so-called self-etch mode.

The adhesive was then applied directly to the tooth surface by means of an applicator (Microbrush applicator, Microbrush International, USA) without acid pretreatment, agitated for approx. 20 s with gentle pressure and then blown with an oil-free air stream to remove the solvent until an immobile film had formed when exposed to the air stream. Subsequently, an LED lamp (Bluephase Style, 1200 mW/cm², 560 nm, Ivoclar Vivadent AG) was used for 10 s to expose the specimen. The test specimen prepared in this way was clamped with the prepared side facing upwards in the clamping device according to EN ISO 29022: 2013. Care was taken to ensure that the adhesive layer was not damaged during the insertion and positioning of the tooth, as well as during the placing of the upper part of the clamping device. Specimens in which the adhesive layer was damaged by premature contact with the top of the clamping device were discarded. The tooth was fixed in the clamping device by slightly tightening the retaining screws. A dental filling composite (Tetric Evo-Ceram BulkFill, Ivoclar Vivadent AG) was then applied through the opening of the clamping device in a layer thickness of 2 to 4 mm. The adaptation pressure applied was selected in such a way that the composite was brought into contact with the adhesive without any bubbles. Excessive pressure leads to leakage of the composite below the clamping device and to the formation of composite beads around the composite cylinder. Test specimens with such annular composite beads were discarded. Subsequently, the composite was polymerized according to the instructions for use (10 s, 560 nm, Bluephase Style polymerization lamp, Ivoclar Vivadent AG).

To measure the shear bond strength, the adhesively bonded composite cylinders were sheared from the tooth at 23° C. using a Zwick-Roell Z010 testing machine (500 N load cell, test speed 1 mm/min) following the Ultradent method (EN ISO 29022, year 2013). The shear bond strength was determined to be 16.2 MPa (dentin) and 19.5 MPa (enamel). These are excellent bond strength values for an enamel-dentin adhesive, which ensure a durable bond between the filling composite and the tooth structure.

### Example 5

Measurement of the Biocompatibility of the Adhesive from Example 4

To measure the biocompatibility of the adhesive from Example 4, the adhesive was light-cured in thin slices, and then the slices were dispersed in water. Cell viability was determined by the WST-1 assay using mouse fibroblasts of cell line LS929. This assay uses the tetrazolium salt WST-1 (4-(3-(4-iodophenyl)-2-(4-nitrophenyl)-2H-5-tetrazolium)-1,3-benzene disulfonate), which is enzymatically converted by viable, metabolically active cells to a red formazan dye whose color intensity is determined photometrically. The assay was designed following the publication by Y. M. Pupo et al (“Cytotoxicity of Etch-and-Rinse, Self-Etch, and Universal Dental Adhesive Systems in Fibroblast Cell Line 3T3”, Scanning Vol. 2017, Article ID 9650420). Monomeric HEMA was used as a positive control. No cytotoxicity was found against mouse fibroblasts.

In addition, genotoxicity was examined in accordance with DIN EN ISO 10993-3 (2018) by means of the COMET assay. The comet assay is based on a gel electrophoretic determination of damaged DNA strands. The extracts of the cured adhesive showed no DNA-damaging effects in the COMET assay and thus no genotoxicity towards mouse fibroblasts.

### Example 6

Preparation of a Secondary Co-Dispersion Based on A_{P} and B_{P} (50% and 30% Neutralization)

Analogously to Example 3, ⅛ of the solution of copolymer A_{P} obtained in Example 1 and ⅛ of the solution of copolymer B_{P} obtained in Example 2 were each weighed separately into a beaker. The two solutions were mixed in a 250 ml round bottom flask with vigorous stirring. To the resulting turbid solution, 400 mg of DMAP dissolved in 12.5 g of demineralized water was added briskly in small portions using a glass pipette. In a second batch, 240 mg DMAP dissolved in 12.5 g demineralized water was added to the resulting turbid solution. The DMAP amounts correspond to neutralization of 50% and 30% of the phosphoric acid groups in copolymer Aₚ, respectively. The acetone was removed from the formed clear solutions on a rotary evaporator at a pressure of about 250 mbar and a water bath temperature of about 40° C. After removal of the organic solvent, a secondary co-dispersion was obtained in each case as milky, viscous dispersions.

Analogous to Example 4, adhesives with a solids content of 30% were prepared with these dispersions by adding CQ, EMBO and ethanol, and the shear bond strength to tooth structure was investigated. Adhesion values of 15.1 MPa (dentin) and 20.5 MPa (enamel) were measured for the 50% neutralized dispersion and 18.1 MPa (dentin) and 21.9 MPa (enamel) for the 30% neutralized dispersion.

1. A dental material comprising at least one copolymer, characterized in that the copolymer is obtainable by
(I) copolymerization of
(a) one or more monomers M_{G} with a polymerizable (meth)acrylate group,
(b) one or more OH group-containing (meth)acrylates M_{F},
(c) one or more strongly acidic adhesive monomers M_{H} and
(d) optionally one or more polymerizable carboxylic acid monomers M_{CS},
(II) reacting the copolymer A₀ obtained in step (I) in a polymer-analogous reaction with at least one functionalized, radically polymerizable monomer M_{P}, which reacts with the OH groups of the monomer M_{F} in the polymer chain of A₀ to form a copolymer A_{P} carrying polymerizable side groups, and
(III) neutralizing the copolymer Aₚ with a base.

2. The dental material according to claim 1, wherein the copolymer A₀ has the following composition:
(a) 20 to 80% by weight of monomer M_{G},
(b) 1 to 70% by weight of monomer M_{F},
(c) 1 to 40% by weight of monomer M_{H} and
(d) 0 to 30% by weight of monomer M_{CS},
where all percentages refer to the mass of copolymer A₀.

3. The dental material according to claim 1, wherein the copolymer Aₚ is obtainable by carrying out the copolymerization of the monomers M_{G}, M_{F}, M_{H} and optionally M_{CS} to copolymer A₀ according to step (I) in an organic solvent, and, after reacting the copolymer A₀ with monomer Mₚ (step (II)) and subsequent neutralization (step (III)),
(IV) optionally adding water to the copolymer solution obtained in step (III) and stirring the solution, and
(V) then optionally distilling the organic solvent off.

4. The dental material according to claim 1, which, in addition to copolymer A_{P}, comprises a second copolymer B_{P} whose composition differs from that of copolymer A_{P}.

5. The dental material according to claim 4, wherein the copolymer B_{P} is obtainable by
(I) copolymerization of
(a) one or more monomers M_{G} with a polymerizable (meth)acrylate group,
(b) one or more OH group-containing (meth)acrylates M_{F},
(c) optionally one or more polymerizable carboxylic acid monomers M_{CS},
(II) reacting the resulting copolymer B₀ in a polymer-analogous reaction with at least one functionalized, radically polymerizable monomer M_{P}, which reacts with the OH groups of the monomer M_{F} in the polymer chain of B₀ to form a copolymer B_{P} carrying polymerizable side groups, and
(III) neutralizing the copolymer Bₚ with a base.

6. The dental material according to claim 4, wherein the copolymer B₀ has the following composition:
(a) 30 to 85% by weight of monomer M_{G},
(b) 5 to 55% by weight of monomer M_{F} and
(c) 1 to 40% by weight of monomer M_{CS},
where all percentages are based on the mass of copolymer B₀.

7. The dental material according to claim 4, wherein the mixture of copolymers A_{P} and B_{P} is obtainable by
preparing a copolymer A₀ by copolymerization of the monomers M_{G}, M_{F}, M_{H} and, optionally, M_{CS} in an organic solvent and converting it into copolymer A_{P} by polymer-analogous reaction with at least one monomer Mₚ,
preparing a copolymer B₀ by copolymerization of the monomers M_{G}, M_{F} and, optionally, M_{CS} in an organic solvent and converting it into copolymer B_{P} by polymer-analogous reaction with at least one monomer Mₚ,
mixing the solutions of copolymer A_{P} and B_{P},
neutralizing the mixture by adding a base,
optionally adding water to the neutralized mixture, and
then optionally removing the organic solvent.

8. The dental material according to claim 1, wherein the amount of the monomer M_{P} is such that
10 to 100 mol % of the OH groups of the monomer building blocks M_{F} in the copolymer A_{P} are reacted with M_{P}, and
optionally 10 to 100 mol % of the OH groups of the monomer building blocks M_{F} in the copolymer B_{P} are reacted with M_{P}.

9. The dental material according to claim 1, in which
the monomer(s) M_{G} is (are) selected from n-propyl methacrylate, neopentyl methacrylate, n-butyl methacrylate, phenyl ethyl methacrylate, n-pentyl methacrylate, n-hexyl methacrylate, cyclohexyl methacrylate, 2-ethylhexyl methacrylate, n-octyl methacrylate, n-dodecyl methacrylate, tetradecyl methacrylate and hexadecyl methacrylate, and the corresponding acrylates and mixtures thereof; and/or
the monomer(s) M_{F} is (are) selected from 2-hydroxyethyl (meth)acrylate, 2-hydroxypropyl (meth)acrylate, 3-hydroxypropyl (meth)acrylate, 4-hydroxybutyl (meth)acrylate and mixtures thereof; and/or
the monomer(s) M_{P} is (are) selected from 2-isocyanatoethyl (meth)acrylate, the addition products of 1 mole of 2-hydroxyethyl (meth)acrylate or 2-hydroxypropyl (meth)acrylate each with 1 mole of a diisocyanate, hexa-methylene-1,6-diisocyanate (HMDI), 2,2,4-trimethyhexamethylene-1,6-diisocyanate (TMDI), isophorone diisocyanate (IPDI), 2-isocyanatoethyl methacrylate (IEMA) and mixtures thereof; and/or
the monomer(s) M_{H} is (are) selected from 2-(meth)acryloyloxypropyl dihydrogen phosphate, 2-(meth)acryloyloxyethyl dihydrogen phosphate, 4-(meth)-acryloyloxybutyl dihydrogen phosphate, 6-(meth)acryloyloxyhexyl dihydrogen phosphate and 10-(meth)acryloyloxydecyl dihydrogen phosphate, vinylphosphonic acid, 4-vinylphenylphosphonic acid, 4-vinylbenzylphosphonic acid, 2-(meth)acryloyloxyethyl phosphonic acid, 2-[4-(dihydroxyphosphoryl)-2-oxa-butyl]-acrylic acid ethyl ester, and mixtures thereof; and/or
the monomer(s) M_{CS} is (are) selected from (meth)acrylic acid, itaconic acid, crotonic acid, maleic acid and fumaric acid and their half esters, 2-(hydroxy-methyl)acrylic acid, 4-(meth)acryloyloxyethyltrimellitic acid, 10-(meth)acryloyloxydecylmalonic acid, 2-(meth)acryloyloxymethylsuccinic acid, 4-vinylbenzoic acid, and mixtures thereof.

10. The dental material according to claim 1, wherein the base for neutralizing the copolymer A_{P} or B_{P} or the mixture of A_{P} and B_{P} is selected from LiOH, KOH, NaOH, NH₄OH, organic amines, ethylamine, n-butylamine, dibutylamine, trimethylamine, tributylamine (TBA), triethylamine (TEA), N-methylmorpholine, dimethylcyclohexyl amine, amino acid derivatives, lysine methyl ester dihydrochloride, arginine hydrochloride, glycine ethyl ester, OH-functionalized amines, methyldiethanolamine, diethylethanolamine, dimethylethanolamine, tris(hydroxymethyl) aminomethane, ethanolamine, diethanolamine, triethanolamine (TEOA), butanolamine, dibutanolamine, 3-dimethylamino-2-propanol (DMAP), 1,3-diamino-2-propanol (DAP), higher molecular weight ether group-containing amines, amino group-terminated oligomeric ethylene oxide, propylene oxide oligomers, and mixtures thereof.

11. The dental material according to claim 1, wherein the base or base mixture is added in an amount of from 20 to 90 mol % based on the content of strongly acidic adhesive monomers M_{H}.

12. The dental material according to claim 1, wherein the copolymer A_{P} and/or B_{P} has a number average molecular weight of from 5,000 to 60,000 g/mol as measured by gel permeation chromatography.

13. The dental material according to claim 1, which comprises
10 to 50% by weight of at least one copolymer A_{P} and optionally B_{P},
50 to 90% by weight dispersion medium and
optionally 10 to 70% by weight of water,
in each case based on the total mass of the dental material, the water content being included in the quantity of dispersion medium, if present.

14. The dental material according to claim 1 for intraoral use in the restoration of damaged teeth, as a dental adhesive or enamel-dentin adhesive.

15. The dental material according to claim 1 for non-therapeutic use as an adhesive in the manufacture or repair of dental restorations.
